(19) United States
(12) Patent Application Publication
(10) Pub. No.: US 2015/0366938 A1
(43) Pub. Date: Dec. 24, 2015

(54) NUTRITIONAL SUPPLEMENT FOR PATIENTS SUFFERING FROM RHEUMATIC DISEASES

(71) Applicant: Héctor de Jesús VÉLEZ-RIVERA, Zapopan, Jalisco (MX)

(72) Inventor: Héctor de Jesús VÉLEZ-RIVERA, Zapopan, Jalisco (MX)

(21) Appl. No.: 14/434,619

(22) PCT Filed: Oct. 11, 2012
(86) PCT No.: PCT/IB2012/002035

§ 371 Date: Sep. 4, 2015

(51) Int. Cl. A61K 38/16; A61K 36/889; A61K 45/06; A23L 1/304; A23L 1/305; A23L 1/30; A23L 1/302; A23L 1/303; A61K 36/02; A23L 1/337

(52) U.S. Cl. CPC A61K 38/168; A61K 36/02; A61K 36/889; A61K 45/06; A23L 1/337; A23L 1/3056; A23L 1/3055; A23L 1/3002; A23L 1/3051; A23L 1/302; A23L 1/303; A23L 1/304; A23L 1/3008; A23V 2002/00

(57) ABSTRACT

The invention relates to a nutritional supplement comprising: a) from 9 to 17 wt. % of Spirulina maxima alga; b) from 20 to 40 wt. % of concentrated whey protein; c) from 10 to 40 wt. % of isolated soy protein; y d) from 20 to 40 wt. % of coconut powder. The pharmaceutical form of said nutritional supplement is a powder for reconstitution, a solution, a suspension, an emulsion, capsules, tablets, flakes or fruit-enriched bars, and can be used to improve the nutritional state of people who suffer from rheumatic diseases.

### TECHNICAL FIELD

The instant invention relates to the preparation of nutritional products, and more particularly, relates to a nutritional supplement for patients with rheumatic diseases.

### BACKGROUND OF THE INVENTION

Rheumatic or joint diseases are a major cause of chronic pain and disability in the developed world, affecting to millions of people worldwide and consuming a lot of economic resources, these diseases cause other problems such as loss of job of patients, loss of independence, reduced number of social interactions, anxiety and a decrease in the welfare of the people who suffered from these diseases.

The prevalence of these diseases increases with factors such as age and even the lifestyle (obesity and lack of physical activity). Therefore, it is expected that with the aging of the world population and the changes that have occurred in that lifestyle over the years, the burden of rheumatic diseases on society will increase dramatically in the coming years.

For example, in the United States of America, according to a study carried out by the Centers for Disease Control and Prevention (CDC) in 2010, an estimated 48 million people in this country suffer from some joint disease, which is an increase of approximately 3 million people in the last three years. In addition, it is also estimated that by 2030, there will be an increase of approximately 40% in the number of people who suffer some form of joint disease, i.e. about 67 million people will affected by joint diseases.

Among the most frequent joint diseases, it can be mentioned rheumatoid arthritis, osteoarthritis, fibromyalgia and gouty arthritis.

Rheumatoid arthritis (RA) is a systemic autoimmune disease with a invariably slow and progressive course characterized by chronic joint inflammation, this disease primarily affects diarthrodial joints in a symmetrical manner, with remissions and exacerbations, the (articular and extra-articular) clinical manifestations vary from very mild to some very intense, some of them are destructive or are mutilating forms.

The etiology of RA is unknown, recent theories suggest that, in a susceptible host, synovial inflammation may be initiated by antigen non-specific mechanisms of innate immunity that subsequently lead to autoimmune responses of adaptive immunity, maintained by antigens articular or systemic.

Currently, there is no cure for RA, so that treatment is only to focus in decrease disease activity with the purpose of minimizing the possibility of joint injury, relieve pain, maintain the better possible performance status and quality of life and finally achieve complete reduction, which is difficult to obtain.

The base of allopathic treatment for RA is the use of “Disease Modifying Antirheumatic Drugs” (DMARDs), which are indicated in the earliest form of the disease, to reach a faster remission in the disease because otherwise it may cause structural damage. While DMARDS achieve remission of the disease, it is necessary to combine them with nonsteroidal anti-inflammatory drugs (NSAIDs) and glucocorticoids.

In addition, there are also exist the complementary medicines, which are used in combination with allopathic medicine to help more effectively treat joint diseases. Currently, data from several studies have refuted speculation that the use of complementary medicines could hinder patients to seek conventional treatments, since it has been found that most patients use complementary medicines simultaneously with orthodox medicine.

Among the complementary medicines, it can be mentioned nutritional supplements. In general, when we refer to nutrition we first in our palate, in our taste for certain foods. However, an increasing percentage of the population know that nutrition is also to provide the body with the nutrients needed to be healthy. In elderly patients with rheumatic or joint diseases, a proper nutrition is essential, in order to have a continued strengthening of muscle, bone and cartilage tissues as well as for the proper functioning of the immune system, that are key factors for a rapid improvement and a good control long-term disease.

For some years, mainly in England, Germany and the United States, there has been a new nutrition concept covering not only diet but also nutritional supplements, which are considered of vital importance in the nutrition of patients with rheumatic diseases.

This new concept is called orthomolecular nutrition, which takes into account that all tissues have a structure that depends on cooperation between different types of cells, and thus is fundamental to the health of tissues and organs that cells are healthy.

Also, nutritional supplements can comprise, between its components, various components of natural origin.

For example, the alga Spirulina maxima alga (spirulina), which was consumed by the Aztecs due to its properties and high nutritional value, is excellent to complement the daily diet, since it has a 65% protein content (vegetable protein), which is even higher than in the fish flesh. It is also very easy to digest, in this regard from 85 to 95% of the alga proteins are assimilated by the body. This is particularly favorable for most elderly with rheumatic diseases, which have a difficulty for digesting complex proteins.

In addition, spirulina contains polyunsaturated fatty acids, which have important functions such as regulating blood pressure and regulate the process of inflammation, polyunsaturated fatty acids are help to take better control of rheumatic disease. Due to its vegetal nature, spirulina does not contain cholesterol and provides a huge amount of Vitamin A and E that are essential for the improvement and control of rheumatic diseases. Spiruline also contains folic acid and several vitamins of B complex, even it has higher contents than those found in cereals, fruits, vegetables and certain seeds. Spiruline also provides iron, calcium, magnesium, chromium, selenium and zinc, which are minerals that have an important role in the control process rheumatic disease. Due that spirulina has high contents of antioxidants (betacarotene, xanthophyll, cryptoxanthin, echinenone, zeaxanthin, lutein, chlorophyll, phycocyanin, phenols, tocopherols) they are favorable reverting the inflammatory process, avoiding pain and stiffness. Spirulina consumption is also especially beneficial in cases of anemia, demineralization and depletion, which are factors that often accompany rheumatic diseases and the elderly, besides helping to restore energy and vitality, and detoxify the body.

On the other hand, the isolated soy protein is a refined form of soy protein with a minimum protein content of 90% on a moisture free basis. The protein is made from defatted soy flour, of which most of its non-protein components, fats and carbohydrates are removed. Due to the amino acid content of the soy protein, it promotes reduction in cholesterol and triglyceride levels and attenuates insulin levels after ingestion and also regulates glucose levels in the blood. Of all legumes, soy is one that has more antioxidants (isoflavone, daidzein, genistein, glycitein), which maintain the immune system in good condition, that is an essential factor to maintain a continuous anti-inflammatory process in the body and thus maintain a prolonged control in the rheumatic disease.

On the other hand, whey protein is a collection of globular proteins that can be physically isolated from whey, which is a byproduct of cheese and dairy products, in turn made from milk from cows, sheep or goat. From the chemical viewpoint, whey protein is a mixture of proteins such as beta-lactoglobulin (65%), alpha-lactalbumin (25%) and serum albumin (8%). Whey has the highest biological value of any known protein, i.e. it becomes a high percentage of muscle protein during the metabolic activities and does not include lactose found in whole milk, which can affect the geriatric patient with rheumatic disease.

Degenerative muscle loss is called sarcopenia, this is common due to sedentary or the progress of age, sarcopenia is a negative element of a rheumatic disease. With the appropriate amount of protein, through the daily diet, the synthesis of muscle tissue and energy production is favored, and the immune system is also improved by favoring the development of intestinal microflora and preventing intestinal inflammation.

Coconut, fruit of Cocos nucifera, has unique features that make it comparable to breast milk as it strengthens the immune system, which is a vital element for the geriatric patient with rheumatic diseases; coconut also it helps to achieve and maintain proper body weight, provides energy and is a source of vitamins and minerals.

Coconut oil, after breast milk is the highest source of nature in medium chain fatty acids (MCT), which increases metabolism and helps in reducing cholesterol levels in blood, has antiviral, antibacterial and antifungal properties, favoring the functioning of the immune system, which is very important for the long term control of rheumatic diseases. It also has protective effects on the heart and liver. In addition, coconut fatty acids stimulate the metabolism by increasing the natural mechanisms of detoxification, repair and cell growth. The coconut contains magnesium that is related with the proper functioning of intestine, nerves and muscles, it is part of bones and teeth and has a mild laxative effect preventing constipation. Coconut oil also contains phosphorus that increases the energy metabolism, in addition to potassium, which is necessary for the transmission and generation of nerve impulses, for normal muscle activity and to maintain the water balance inside and outside the cell. Coconut oil contains 30% of fiber recommended for daily consumption, and prevent constipation and prevent colon cancer.

Also, in the prior art, it is possible to find various nutritional supplements for patients suffering from joint diseases.

For example, European Patent No. EP 1407777 describes an agent comprising Hydrangea macrophylla Seringe var. Thunbergii Makino or Hydrangeae Dulcis Folium, or an extract thereof as an active ingredient, the agent may contain other active ingredients for the prevention or treatment of arthritis as well as excipients and elements such as vitamins, minerals, etc. This agent is administered orally and is useful for treating or preventing rheumatoid arthritis. Furthermore, this agent can be used in food or drink, and in animal feed. When used as animal feed may contain, among other ingredients, Spirulina and coconut paste. The agent or formulation may be prepared as tablets, powders, granules, etc., which are suitable for oral administration.

In addition, US Application Patent No. 2006/0281822 discloses a method for reducing levels of homocysteine, which is an amino acid that is related with diseases such as rheumatoid arthritis in an individual. To do this, a food supplement containing dimethyl sulfone as active ingredient is administered. In addition, the nutritional supplement may comprise additional ingredients such as Spirulina, soy protein and whey, coconut oil, vitamins, minerals, etc., in combination with various types of excipients, including flavorings. The supplement may be in the form of capsules, tablets, powders, liquids, etc., and it may be administered by various routes, preferably orally. The purpose of this formulation is to maintain homocysteine levels in the patient, which can prevent or treat caused or exacerbated by elevated levels of homocysteine diseases.

Also, International Publication No. WO 2008/153426 discloses a composition with anti-inflammatory activity and their use to treat or prevent various diseases including joint diseases. The active ingredient of said composition consists of a mixture of omega 3 fatty acids extracted from marine oils. The composition may further contain other anti-inflammatory components, such as simple sugars, vitamins, soy protein and spirulina, as well as vegetable oils such as coconut oil. The composition may include excipients, and may be in a form of a tablet, pill, capsule, powder, granules, or any form that can be added to a food or beverage.

According to the above, in the state of the art there are dietary supplements that are used to improve the nutritional status of patients suffering from joint diseases. However, none of them discloses or suggests a nutritional supplement such as that of the present invention that is used as an alternative to effectively improve the quality of life of these patients to improve and maintain optimal nutritional status of the same.

### OBJECTS OF THE INVENTION

Taking into account the drawbacks of the prior art, an object of the present invention is to provide a nutritional supplement for patients suffering from rheumatic diseases, capable of providing nourishment to the body cells of these patients that need energy in order to properly maintain its structure and function.

A further object of the present invention is to provide a nutritional supplement for patients suffering from rheumatic diseases that would achieve a significant improvement in the nutritional status of these patients.

Another object of the present invention is to provide a nutritional supplement for patients suffering from rheumatic diseases, which allows significantly improving a significant improvement against the degenerative process that is present in rheumatic diseases.

### SUMMARY OF THE INVENTION

In order to accomplish the above objects, it has been created a nutritional supplement comprising: a) Spirulina maxima alga; b) concentrated whey protein; c) isolated soy protein; and d) coconut powder. In a preferred embodiment of the invention, the nutritional supplement comprises: a) from 9 to 17% by weight of Spirulina maxima alga; b) from 20 to 40% by weight of concentrated whey protein; c) from 10 to 40 wt % of isolated soy protein; and d) from 20 to 40% by weight of coconut powder.

Other aspects of the invention consider the use of nutritional supplement to improve the nutritional status of patients suffering from any rheumatic disease.

### DETAILED DESCRIPTION OF THE PREFERRED EMBODIMENTS

As it was previously mentioned, there is need for nutritional supplements that help to improve the nutritional aspect of patients suffering from rheumatic diseases.

Thus, it has been found that a nutritional supplement comprising: a) alga Spirulina maxima; b) concentrated whey protein; c) isolated soy isolate; and d) coconut powder, can be used to improve the nutritional status of patients suffering from rheumatic or articulate diseases, since it contains all the nutritional groups necessary for this purpose.

Consequently, in a particularly specific embodiment of the present invention, a nutritional supplement for improving the nutritional status of patients suffering a rheumatic disease is disclosed, the supplement comprises

a) from 9 to 17% by weight of Spirulina maxima alga;

b) from 20 to 40% by weight of concentrated whey protein;

c) from 10 to 40% by weight of isolated soy protein; and

d) from 20 to 40% by weight of coconut powder.

More preferably, the nutritional supplement of the present composition comprises:

a) from 11 to 15% by weight of Spirulina maxima alga;

b) from 25 to 35% by weight of concentrated whey protein;

c) from 20 to 30% by weight of isolated soy protein isolate; and

d) from 25 to 35% by weight of coconut powder.

In another embodiment of the present invention, the nutritional supplement optionally comprises an effective amount of one or more of the following components: essential amino acids, nonessential amino acids, vitamins, minerals, fatty acids, natural pigments, coenzyme Q10, routine, Chlorella alga, phytoestrogens, natural or artificial flavors and honey.

Preferably, the essential amino acids are selected from the group comprising leucine, threonine, phenylalanine, isoleucine, valine, lysine, histidine, methionine and tryptophan.

Nonessential amino acids are selected from the group comprising glutamic acid, aspartic acid, proline, serine, alanine, tyrosine, arginine, cysteine and glycine.

Vitamins are selected from the group comprising inositol, vitamin A, vitamin B1, vitamin B2, vitamin B3, vitamin B12, vitamin E, vitamin C, vitamin D6, pantothenic acid, biotin and folacin.

Regarding minerals, they are selected from the group comprising potassium, sodium, phosphorus, calcium, magnesium, iron, manganese, zinc, copper and selenium.

With respect to the fatty acids, they are selected from the group comprising palmitic acid, gamma linoleic acid, linolenic acid, palmitoleic acid, oleic acid, stearic acid, heptadecanoic acid and myristic acid.

Finally, the natural pigments are selected from the group comprising fitocianina, chlorophyll, carotenoids and anthocyanins.

The nutritional supplement of the present invention is orally administered and is prepared in a dosage form selected from powders for reconstitution, solutions, suspensions, emulsions, capsules, tablets, flakes and bars enriched with fruit, preferably is prepared as a powder to be reconstituted in water or juices.

This nutritional supplement is used as an alternative to effectively improve the quality of life of patients with a rheumatic disease, since the supplement improves and maintains an optimal nutritional status of the patients.

The present invention will be better understood from the following examples, which are included for illustrative purposes in order to allow the understanding of the preferred embodiments of the instant invention, these examples do not imply that there are not other forms not illustrated that can be carried out based on the above detailed description.

### EXAMPLES

### Example 1

### Preparation of the Nutritional Supplement

In order to prepare the nutritional supplement in a powder form for reconstitution, identification and weighing of each of the components of the formulation (Spirulina maxima alga, concentrated whey protein, isolated soy protein and coconut powder) was carried out, which were previously evaluated and approved concerning their physicochemical characteristics.

Subsequently, each component was sized in order to homogenize the particle size, the components were added to a mixing equipment, which was operated in a given speed and time.

After the mixing time concluded, the powder mixture was allowed to stand for 5 minutes to obtain a homogeneous mixture according to an analysis of uniformity mixing, finally the conditioning of the product was carried out in vials of suitable capacity according to the presentation or in single-dose sachets.

For the preparation of solid pharmaceutical forms, they are carried out specific manufacturing processes known in the prior art from the process above described.

### Example 2

### Clinical Tests

To determine aspects of quality of life as well as nutritional and anthropometric changes in elder with rheumatoid arthritis who incorporated the nutritional supplement of the present invention to their diet; a pilot and randomized study was developed with two parallel groups of 200 patients with rheumatoid arthritis each. The first group was treated with the nutritional supplement of the present invention in a powder form for reconstitution, and the second with a placebo powder. This study was conducted at the Center for Rheumatic Diseases called “Artricenter” located in Mexico City.

The main inclusion criteria for the clinical test were patients of “Artricenter” aged between 60 and 70 years, male or female, with clinical diagnosis of rheumatoid arthritis pain greater than or equal to 5 on a horizontal visual analog scale (VAS) from 0 to 10.

Exclusion criteria were: patients suffering from an inflammatory disease other than rheumatoid arthritis, people with steroid injection until two months before entering to the protocol, people with diabetes mellitus and people consuming drugs or alcohol.

In addition, patients who did not attend either control or treatment or patient who decided to leave by their own were eliminated from the study.

The study lasted three months and the evaluation criteria were socioeconomic status; pathological characteristics, habits, general conducts and current situation of the senses, nutritional assessments with anthropometric measurements and evaluation of fragility.

After the initial assessment, the selected patients were assigned with a random number by which they were placed into two equal groups

The nutritional supplement group (group 1) received 3 bottles each with 900.0 g of the nutritional powder to prepare a shake. The powder contained Spirulina maxima alga, concentrated whey protein, isolated soy protein and coconut powder to administer daily 30 grams of powder, the shake was prepared by the addition of water under stirring. The shake was drunk in the morning before breakfast for 3 consecutive months.

The group of the placebo powder (Group 2) received 3 bottles each with 900.0 grams of a similar powder, regarding appearance and consistency, to the nutritional supplement, but containing a suspending agent and flavoring instead of the active components of interest. The powder (30 grams) was used to prepare a shake by adding water under stirring, which was administered daily in the morning before breakfast for 3 consecutive months.

The individuals of both groups were compared in terms of age, gender, weight, size and characteristics of pain; no statistically significant differences between them were observed; therefore they were considered homogeneous populations (Table 1).

TABLE 1
Comparison of the groups treated with Dietary Supplement
Powder (group 1) and Placebo Powder (group 2) by
demographic and clinical characteristics.

| | | GROUP 1 | GROUP 2 | p Value |
|---|---|---|---|---|
| Age | | 67.5 (60.0-75.0) | 68.05 (64.0-72.1) | 0.3021* |
| Gender | Men | 0.42 | 0.37 | — |
| | Women | 0.58 | 0.63 | |

| | | | |
|---|---|---|---|
| Weight | 72.77 | 73.12 | 0.521** |
| Height | 142.01 | 150.16 | 0.715** |

*Obtained by T student.
**Obtained by Chi². Confidence intervals at 95%

At the beginning, in Group 1, 72% of patients perceived themselves ill in the last three months, while at the end of the study was reduced to 47% of patients who perceived ill. In the case of patients with pathological conditions such as gastritis and constipation, an improvement of 27 and 21% respectively was perceived.

Similarly, in the same group, at beginning, 68.5% consumed at least one medicine and 24.5% consumed more than 3 medicines. At the end of the study, these percentages were reduced to 57.3% of patients who consumed at least one drug and 15.1% of patients who consumed more than 3 medicaments.

Also, to evaluate the perception of the condition of the senses in the patients in group 1, 99% of the study population mentioned that they were able to distinguish flavors, while most older adults (96.5%) stimulates food smell appetite; regarding auditory perception, 93.1% listen well and 62% reported an improvement in their vision. These results indicate an increase in the general perception of the senses according to the comparison with the data measured at the begging of the study, when 85% of the population distinguished flavors, 86.3% mentioned that sniffing food stimulated their appetite, while 90% listened well and 50% reported having good vision.

With respect to nutritional data, 81.2% of patients in group 1 presented a normal nutritional status, while 17.84% were at risk of malnutrition and 0.96% in malnutrition. In assessing the nutritional status of patients at baseline, 2.15% of them were found in a state of malnutrition and more than half (52.12%) at risk of suffer malnutrition, while the remaining 45.73% had a normal nutritional status.

The results of the final evaluation after the nutritional intervention are shown in Table 2.

TABLE 2
nutritional status of patients of group 1 before and after the clinical test.

| | No. of patients | No. of patients | |
|---|---|---|---|
| | at the beggining | at the end | Nutritional |
| Nutritional Status | of the test. | of the test. | change % |
| Normal | 92 | 162 | 176.0 |
| At risk of malnutrition | 104 | 36 | 65.0 |
| Malnutrition | 4 | 2 | 50.0 |

Regarding Group 2, while assessing the nutritional status of patients at beginning, it was found that 0.68% of them were malnourished, 11.5% were at risk of suffering malnutrition and the remaining 87.3% were in normal nutrition. After 3 months of the study, it was found that 88.16% of patients had a normal nutritional status, 11.18% were at risk of malnutrition and 0.66% were in malnutrition. Data are shown in Table 3.

TABLE 3
Nutritional status of patients in group 2 before and after the clinical test

| | No. of patients | No. of patients | |
|---|---|---|---|
| | at the beginning | at the end | Nutritional |
| Nutritional Status | of the test. | of the test. | change % |
| Normal | 175 | 175 | 0 |
| At risk of malnutrition | 24 | 23 | 4.16 |
| Malnutrition | 1 | 1 | 0 |

With respect to the anthropometric data at baseline patients in group 1 had a mean weight of 72.7±11.6 kg for males and 62.4±13.7 kg for the female gender, and a body mass index (BMI) of 27.2±3.8 for male and 28.4±5.8 for the female gender. At the end of the study, these patients in group 1 had a mean weight of 73.3±11.5 kg for males and 62.9±13.6 kg for the female gender, and a BMI of 27.6±4.3 for male patients and 28.8±5.8 for the female gender. In the case of patients of group 2, no meaningful changes were evident.

With regard to the mass reserve according to arm circumference, the average was 28.8-29.7 cm, wherein 52.6% of the population was in the average according to age, 8.7% were above the average and 15.8% below the average, such measures were carried out at the end of the study.

On the other hand, the study regarding the frequency of fragility (according to the Barber's scale) in patients of group 1; 70.9% was found in the initial phase; at the end of the study this syndrome of fragility decreased to 50.5% in both genders.

Also, the results obtained at the beginning and the end of the study for patients in group 1 were the following, for the female gender patients without fragility were 24.4% and 35.7%, respectively, for males, patients without fragility were 8.7% and 13.8%, respectively.

It is worth mentioning, that there is no consensus on the concept of fragility, however, there is consensus in considering fragility as pre disability or a situation of vulnerability in the elderly, fragility is multifactorial (physiological, hormonal, sarcopenia, swelling, etc.), fragility is the result of the diminishing of the physiologic reserve with an increased risk of dependency and a greater likelihood of adverse health events.

In relation to nutritional data of the assessments that were carried out, it can be concluded that patients in Group 1 who received the supplement of the present application improved their nutritional status, going from a state of malnutrition to a level at risk of malnutrition, and from of malnutrition to a normal level; in addition it was observed changes in the weight and height data, that is different to group 2 that received the placebo powder.

This test shows a high prevalence of fragility in the elderly evaluated before starting the study; however, the results of the assessments carried out at the end of the study show that, in group 1, fragility conditions of patients decreased, while in group 2 showed no difference in fragility.

Also, the presence of diseases was determined indirectly by asking patients if they took drugs and which daily dosage form. An important result in this study is the disappearance of constipation who reported patients in group 1, highlighting the importance of eating fiber in the nutritional supplement, this is different for group 2 that used placebo powder in which no such effect was observed.

It is also important to highlight the positive change in the diseases referred to during the administration of the nutritional supplement of the present invention, since at the end of the study, patients denied aggravation of symptoms, relapse or interconsultations by the same conditions. In this regard, although the physiological aging is accompanied by a gradual and progressive physical dependence, a proper nutritional intervention in the context of physical activity, will maintain a better state of health for the elderly, thus reducing the onset or aggravation of diseases.

In accordance with the above described, it will be observed that the nutritional supplement for patients with rheumatic diseases has been designed to effectively improve the quality of life of patients with these type of diseases, because the supplement improves and maintains an optimal nutritional status of the patients, and will be apparent to one skilled in the art that the embodiments of the nutritional supplement for patients with rheumatic diseases, as described above, are only illustrative but not limitative of the present invention, since many changes are possible regarding in their details without departing from the scope of the invention, such as the concentration of components as well as the pharmaceutical form used for administration to a patient.

Therefore, the present invention should not be considered as restricted except as required by prior art and by the scope of the appended claims.

1. A nutritional supplement comprising:
a) from 9 to 17% by weight of Spirulina maxima alga;
b) from 20 to 40% by weight of concentrated whey protein;
c) from 10 to 40% by weight of isolated soy protein; and
d) from 20 to 40% by weight of coconut powder.

2. (canceled)

3. A nutritional supplement according to claim 1, comprising
a) from 11 to 15% by weight of Spirulina maxima alga;
b) from 25 to 35% by weight of concentrated whey protein;
c) from 20 to 30% by weight of isolated soy protein; and
d) from 25 to 35% by weight of coconut powder.

4. A nutritional supplement according to claim 1, further comprising an effective amount of one or more of the following components selected from the group comprising: essential amino acids, nonessential amino acids, vitamins, minerals, fatty acids, natural pigments, coenzyme Q10, rutin, Chlorella alga, phytoestrogens, natural or artificial flavors and honey.

5. A nutritional supplement according to claim 4, wherein the essential amino acids are selected from the group comprising leucine, threonine, phenylalanine, isoleucine, valine, lysine, histidine, methionine and tryptophan.

6. A nutritional supplement according to claim 4, wherein the non-essential amino acids are selected from the group comprising glutamic acid, aspartic acid, proline, serine, alanine, tyrosine, arginine, cysteine and glycine.

7. A nutritional supplement according to claim 4, wherein the vitamins are selected from the group comprising inositol, vitamin A, vitamin B1, vitamin B2, vitamin B3, vitamin B12, vitamin E, vitamin C, vitamin D6, pantothenic acid, biotin and folacin

8. A nutritional supplement according to claim 4, wherein minerals are selected from the group comprising potassium, sodium, phosphorus, calcium, magnesium, iron, manganese, zinc, copper and selenium.

9. A nutritional supplement according to claim 4, wherein the fatty acids are selected from the group comprising palmitic acid, gamma linoleic acid, linolenic acid, palmitoleic acid, oleic acid, stearic acid, heptadecanoic acid and myristic acid.

10. A nutritional supplement according to claim 4, wherein the natural pigments are selected from the group comprising fitocianine, chlorophyll, carotenoids and anthocyanins.

11. A nutritional supplement according to claim 4, wherein the supplement is in a dosage form selected from a powder for reconstitution, solution, suspension, emulsion, capsules, tablets, bars and flakes enriched with fruit, preferably a powder to be reconstituted in water or juices, preferably a powder for reconstitution.

12. Use of the nutritional supplement of claim 1 to improve the nutritional status of patients with rheumatic diseases.
